(19) United States
(12) Patent Application Publication
(10) Pub. No.: US 2017/0166054 A1
(43) Pub. Date: Jun. 15, 2017

(54) USING BIOSENSORS TO PREVENT IMPAIRED DRIVING

(71) Applicant: Continental Automotive Systems, Inc., Auburn Hills, MI (US)

(72) Inventors: Eduardo Ayala Rodriguez, Tlaquepaque (MX); Eduardo Gonzalez Alfaro, Tlaquepaque (MX); Adan Efrain Gonzalez Jaime, Tlaquepaque (MX); Jaime Arechiga Chavez, Tlaquepaque (MX)

(21) Appl. No.: 14/967,830

(22) Filed: Dec. 14, 2015

(51) Int. Cl. B60K 28/06; A61B 5/07; A61B 5/11; A61B 5/0402; A61B 5/0476; A61B 5/18; A61B 5/00

(52) U.S. Cl. CPC B60K 28/063; B60K 28/066; A61B 5/18; A61B 5/076; A61B 5/6801; A61B 5/6846; A61B 5/0402; A61B 5/0476; A61B 5/7282; A61B 5/0077; A61B 5/1103; A61B 5/1128; A61B 5/6893; A61B 2010/0009

(57) ABSTRACT

A biosensor is configured to detect an impaired-driving condition of a driver of a vehicle; a biosensor reader, in the vehicle, is configured to wirelessly receive information from the biosensor regarding whether the driver of the vehicle is in an impaired-driving condition; an ignition system of the vehicle is configured to be disabled in response to the biosensor reader receiving information from the biosensor indicating that the driver of the vehicle is in an impaired driving condition; and a telematics unit is configured to wirelessly notify a remotely located third party that the driver is impaired.

### BACKGROUND

Embodiments of the invention relate generally to using one or more biosensors to prevent a vehicle from being driven when a driver's ability to drive is impaired because the driver is drunk or experiencing any other condition that impairs the driver's driving ability.

According to research analysis conducted in the United States, almost 30 people die each day in motor vehicle crashes that involve an alcohol-impaired driver. This amounts to one death every 48 minutes.

Similarly, a driver's driving ability may be impaired by conditions other than intoxication, including, but not limited to, a heart attack, falling asleep, an epileptic seizure, fainting, and the like.

There is a need, therefore, for better ways to prevent driving while a driver's driving ability is adversely affected by alcohol or any other drug or condition that impairs driving ability.

### BRIEF SUMMARY

In accordance with embodiments of the invention, a biosensor is configured to detect an impaired-driving condition of a driver of a vehicle; a biosensor reader, in the vehicle, is configured to wirelessly receive information from the biosensor regarding whether the driver of the vehicle is in an impaired-driving condition; an ignition system of the vehicle is configured to be disabled in response to the biosensor reader receiving information from the biosensor indicating that the driver of the vehicle is in an impaired driving condition; and a telematics unit is configured to wirelessly notify a remotely located third party that the driver is impaired.

### BRIEF DESCRIPTION OF THE DRAWINGS

FIG. 1 is a schematic diagram showing a system having a biosensor for preventing impaired driving in accordance with an embodiment of the invention.

FIG. 2 is a flow chart showing a process for using a biosensor to prevent an impaired driver from operating a vehicle in accordance with embodiments of the invention.

### DETAILED DESCRIPTION

In accordance with embodiments of the invention, an implantable biosensor may be implanted in the driver's body (in-vivo). In some embodiments, the biosensor may sense a driver's blood alcohol level, and then the sensor may wirelessly transmit blood-alcohol-concentration information to a processor within the vehicle. The processor may then determine whether to enable or disable the vehicle's ignition based on the information received from the biosensor.

A biosensor is a device that uses biological molecules, especially enzymes or antibodies, to detect the presence of chemicals. In some embodiments of the invention, a biosensor may be used to detect an analyte (i.e., a substance whose chemical constituents are being identified and measured), such as, for example, alcohol, glucose, and the like. A biosensor may combine a biological component with a physicochemical (i.e., of or relating to physics and chemistry or to physical chemistry) detector for detecting enzymes, antibodies, and the like.

In some embodiments, an implantable sensor may comprise multiple sensors, a coil for wireless power, and miniaturized electronics for wireless communication. The implantable sensor may be covered with an enzyme that reacts with chemicals in the body to measure those chemicals. Information about the sensed chemicals may then be sent wirelessly to a head unit, telematics unit, or any other suitable processor of the vehicle. A user's smartphone, or any other suitable device with wireless communication capability, may be used to receive sensed information from the implantable sensor and then relay that information to the vehicle.

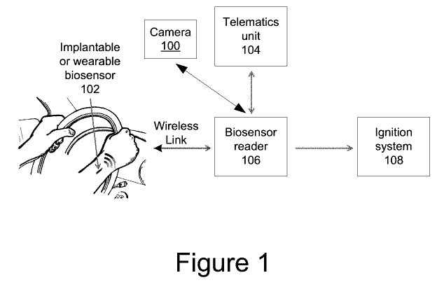

FIG. 1 is a schematic diagram showing a system having a biosensor for preventing impaired driving in accordance with embodiments of the invention. An implantable biosensor 102 is shown implanted into a driver's wrist, as an example. The implantable biosensor could be implanted elsewhere in the driver's body. In another embodiment, a biosensor may be worn on a driver's body, instead of implanted in the driver's body. For example, sensors such as EEG (electroencephalogram for measuring the electrical activity of the brain), EKG (electrocardiogram, which checks for problems with the electrical activity of a person's heart), and the like may be placed on a driver's body.

As another example, SCRAM, or Secure Continuous Remote Alcohol Monitor, which performs transdermal alcohol testing, may be worn on the ankle or wrist and detects alcohol excretion from the skin by sampling the user's sweat and measuring the user's blood alcohol concentration in a manner similar to a conventional breathalyzer test.

A vehicle may have a biosensor reader 106, which may be located: in the steering wheel, in the driver's door, in the driver's seat, or any other suitable location within the vehicle.

The biosensor may communicate wirelessly with the biosensor reader 106 or other module within the vehicle. The implantable biosensors 102 may support wireless technologies, including, but not limited to: RFID, UWB, Zigbee, etc., thereby, allowing the system to support a variety of applications, such as, data transfer, including a unique identifier, from the implantable biosensor 102 to the vehicle. Such a unique identifier may be used to identify the person into which the biosensor has been implanted or who is wearing a wearable biosensor.

Additional wireless protocols may also be used, including, but not limited to: ANT and ANT+, which are proprietary wireless sensor network technologies used in the collection and transfer of sensor data. As a type of personal-area network (PAN), ANT's primary applications include sports, wellness, and home health. For example, ANT is used in heart-rate monitors, speedometers, calorimeters, blood pressure monitors, position tracking, homing devices, and thermometers. Typical radios are built into sports watches and equipment like workout machines. The technology divides the 2.4-GHz industrial, scientific, and medical (ISM) band into 1-MHz channels. The radios have a basic data rate of 1 Mbit/s. A time division multiplexing (TDM) scheme accommodates multiple sensors. ANT+ supports star, tree, mesh, and peer-to-peer topologies. Bluetooth is another PAN technology. The Bluetooth Special Interest Group (SIG) manages the standard. IEEE 802.15.1 also covers it. Bluetooth Low Energy targets health and medical applications. Wi-Fi is the commercial name of the wireless technology defined by the IEEE 802.11 standards. Wi-Fi is a well-known and widespread wireless technology.

Implantation of the biosensor may be performed by personal choice or pursuant to a court order as punishment for recurrent drunk-driving convictions.

The vehicle's head unit, or any other module within the vehicle with access to the information received from the biosensor, may transfer the biosensor information to a telematics unit 104, which in turn may transfer the data to third parties (e.g. police, ambulance, family, etc.).

In some embodiments, a video or infrared camera 100 may be oriented toward a vehicle's driver and may be used for monitoring the driver's eye movements, rate of blinking, and the like. Such information may be used, for example, to determine when the driver is falling asleep. Drowsiness is associated with a decrease in core body temperature and an increase in plasma melatonin, which may be monitored by the implantable biosensor, and a decrease in eye blink rate and an increase in slow eye movement, which may be monitored by the camera 100.

The processor 106 may communicate with an ignition system 108 of the vehicle. As described in more detail below, the ignition system 108 may be disabled, thereby preventing the driver from starting the vehicle, if the biosensor 102 transmits information indicating that the driver's driving ability is impaired.

The biosensor reader 106 may communicate information indicating that the driver's driving ability is impaired to a telematics unit 104, which may, in turn, wirelessly communicate that information, via a cellular network, for example, to one or more third parties, such as family members, emergency responders, law enforcement officers, and the like.

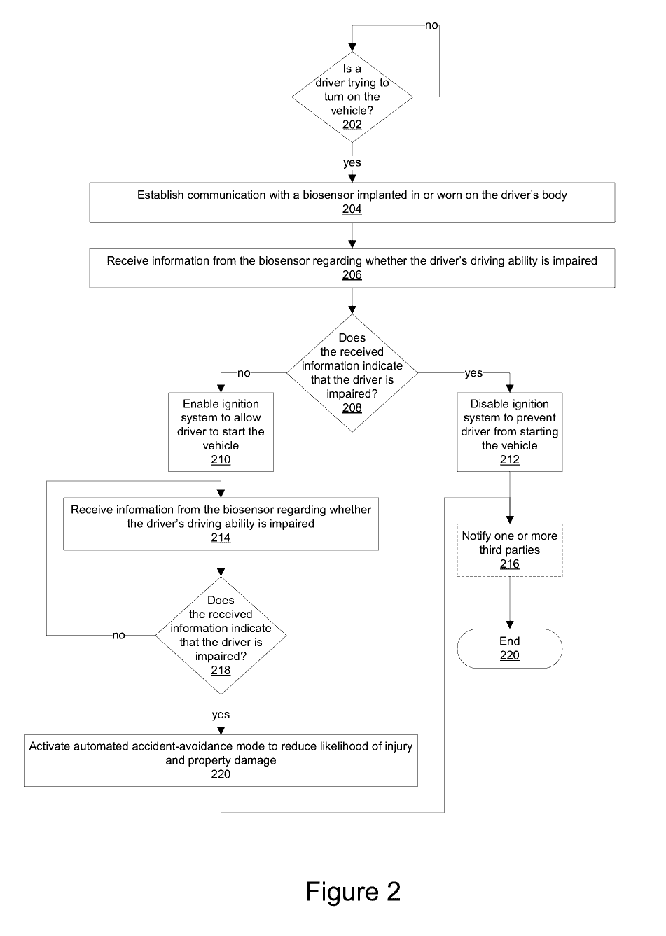

FIG. 2 is a flow chart showing a process for using a biosensor to prevent an impaired driver from operating a vehicle in accordance with embodiments of the invention. Processing begins at 202 and stays there until a determination is made that a driver is trying to turn on the vehicle. At 204, communication is established with a biosensor implanted in or worn on the driver's body and a unique identifier of the biosensor may be received and processed. At 206, information is received from the biosensor regarding whether the driver's driving ability is impaired. Such impairment may take several forms, including, but not limited to: being under the influence of alcohol or other drugs, or experiencing a medical condition that would impair the driver's driving ability.

A determination is made at 208 regarding whether the information received from the biosensor indicates that the driver's driving ability is impaired. If the driver's driving ability is impaired, the vehicle's ignition system is disabled at 212 to prevent the driver from starting the vehicle. Optionally, at 216, one or more third parties may be notified of the driver's impairment. If the driver's driving ability is not impaired, the vehicle's ignition system is enabled at 210 to allow the driver to start the vehicle.

At 214, information is received from the biosensor regarding whether the driver's driving ability is impaired. Such impairment may take several forms, including, but not limited to: being under the influence of alcohol or other drugs, or experiencing a medical condition that would impair the driver's driving ability.

Various types of impaired driving ability may be monitored, including, but not limited to impairment caused by: alcohol, Benzodiazepines (e.g., Valium), Marijuana, other so-called recreational drugs; Diabetes/Hyopoglycemia/blood glucose abnormalities; drowsiness; a seizure; loss of consciousness; and a medication dose either taken or missed.

A determination is made at 218 regarding whether the information received from the biosensor indicates that the driver's driving ability is impaired. If the driver's driving ability is not impaired, processing returns to 214.

If the driver's driving ability is impaired, any of the vehicle's automated accident-avoidance modes may be activated to reduce the likelihood of injury and property damage that could otherwise be caused by the driver's impaired driving ability. For example, various sensors, such as one or more cameras, radar sensors, lidar sensors, infrared sensors, and the like, and/or position information, such as GPS coordinates, of the vehicle may be used to cause the vehicle to come to a stop in a safe area (e.g., on the shoulder of a highway, or in a parking lot off of the road that the vehicle was travelling on). Other suitable action may also be taken such as activating the vehicle's flashing hazard lights.

Optionally, at 216, one or more third parties may be notified of the driver's impairment. And processing ends at 220.

In accordance with embodiments of the invention, impaired drivers are prevented from driving without the driver having to perform a task, such as taking a breathalyzer test before starting the vehicle. A driver will not typically be able to circumvent the system by tampering with it. And impaired drivers will be prevented from driving thereby enforcing laws that are intended to prevent impaired drivers from driving. If and when a driver's driving ability becomes impaired while driving, the impairment may be sensed, and the vehicle may be brought to a safe stop automatically.

1. An apparatus comprising:
a biosensor configured to detect an impaired-driving condition of a driver of a vehicle;
a biosensor reader, in the vehicle, the biosensor reader being configured to wirelessly receive information from the biosensor regarding whether the driver of the vehicle is in an impaired-driving condition;
an ignition system of the vehicle that is configured to be disabled in response to the biosensor reader receiving information from the biosensor indicating that the driver of the vehicle is in an impaired driving condition; and
a telematics unit configured to wirelessly notify a remotely located third party that the driver is in an impaired-driving condition.

2. The apparatus of claim 1, wherein the biosensor is configured to be implanted in the driver's body.

3. The apparatus of claim 1, wherein the biosensor is configured to be worn on the driver's body.

4. The apparatus of claim 3, wherein the biosensor is selected from the group consisting of: one or more electroencephalogram sensors configured to measure the electrical activity of the brain, one or more electrocardiogram sensors configured to measure electrical activity of the driver's heart, and a secure continuous remote alcohol monitor configured to perform transdermal alcohol testing.

5. The apparatus of claim 1, wherein the impaired-driving condition is selected from the group consisting of: intoxication, heart attack, falling asleep, epileptic seizure, and fainting.

6. The apparatus of claim 1, further comprising a camera in communication with the biosensor reader and oriented so as to capture video of the driver's eyes to enable drowsiness detection based on analysis of activity of the driver's eyes.

7. A method comprising:
using a biosensor to detect an impaired-driving condition of a driver of a vehicle;
in the vehicle, wirelessly receiving information from the biosensor regarding whether the driver of the vehicle is in an impaired-driving condition;
disabling an ignition system of the vehicle in response to receiving information from the biosensor indicating that the driver of the vehicle is in an impaired driving condition; and
wirelessly notifying a remotely located third party that the driver is in an impaired-driving condition.

8. The method of claim 7, wherein the biosensor is configured to be implanted in the driver's body.

9. The method of claim 7, wherein the biosensor is configured to be worn on the driver's body.

10. The method of claim 9, wherein the biosensor is selected from the group consisting of: one or more electroencephalogram sensors configured to measure the electrical activity of the brain, one or more electrocardiogram sensors configured to measure electrical activity of the driver's heart, and a secure continuous remote alcohol monitor configured to perform transdermal alcohol testing.

11. The method of claim 7, wherein the impaired-driving condition is selected from the group consisting of: intoxication, heart attack, falling asleep, epileptic seizure, and fainting.

12. The method of claim 7, further comprising: using a camera oriented toward the driver's eyes to capture video for use in performing drowsiness detection based on activity of the driver's eyes.
